(19) United States
(12) Patent Application Publication
(10) Pub. No.: US 2010/0190213 A1
(43) Pub. Date: Jul. 29, 2010

(54) METHOD AND APPARATUS FOR CONTROLLING THE TEMPERATURE OF REACTION VOLUMES

(75) Inventors: Graham Maurice Miller, Dundee (GB); Christopher George Hamilton Courtney, Dundee (GB); Christopher Andrew Williams, By Cupar (GB); John Gordon Freeman, Perth (GB)

Correspondence Address: NIXON PEABODY, LLP, 401 9TH STREET, NW, SUITE 900, WASHINGTON, DC, 20004-2128, US

(73) Assignee: PHOTONASE LIMITED, Dundee (UK)

(21) Appl. No.: 12/376,734

(22) PCT Filed: Aug. 8, 2007
(86) PCT No.: PCT/GB07/03000

§ 371 Date: Mar. 26, 2009

(30) Foreign Application Priority Data
Aug. 11, 2006 (GB) 0616106.1

(51) Int. Cl. C12P 19/34; C12M 1/00

(52) U.S. Cl. 435/91.2; 435/303.1

(57) ABSTRACT

A method and apparatus for controlling the temperature of one or more reaction volumes contained in respective reaction vessel(s) through absorption of electromagnetic energy by a reactant medium of the reaction volume(s). The method comprises providing one or more-reaction volumes (1) in respective reaction vessel(s) (3) such. that the reaction volume(s) each have a given depth; providing at least one source of electromagnetic radiation having selected spectral characteristics; directing the electro-magnetic radiation (2) into each of the one or more reaction vessel(s) to pass along an absorption path through the reaction volume(s); selecting the spectral characteristics of the electromagnetic radiation for providing substantially uniform energy absorption by the reactant medium along the full length of said absorption path; and providing the absorption path for each of the one or more reaction volumes to be greater than or equal to a substantial proportion of a width of that reaction volume in a direction perpendicular to the depth thereof.

The present invention relates to a method and apparatus for controlling the temperature of reaction volumes.

The need to accurately control the temperature of reaction volumes during analytical procedures such as PCR is well understood.

The Polymerase Chain Reaction (PCR) is a bio-analytical procedure which uses enzymes and other reagents to amplify DNA or RNA from undetectable amounts to detectable amounts through a thermal cycling technique. Each thermal cycle produces a doubling of the target DNA sequence resulting in an exponential accumulation of this sequence.

Typically, temperature control of the reaction volumes is achieved using apparatus which relies on thermal conduction through the walls of the vessel used to contain the reaction volumes, aided by convection within the reaction volume itself.

However, indirect heating of an arbitrary reaction volume using conduction and consequent convection is relatively inefficient. This is because large thermal masses are used to transfer heat to relatively small reaction volumes. As a result, time is spent thermally cycling the large thermal masses and then allowing them to come into equilibrium with the small thermal mass of the reaction volumes. This means that the overall process time for a procedure can be undesirably lengthy, particularly in procedures involving a large number of thermal cycles.

One way of decreasing the overall process time that has been proposed is to reduce the reaction volume to microscopic quantities, which results in an increase in the rate of conduction heating and cooling.

However, for the purposes of post amplification processing, non-microscopic quantities are generally required. For this reason, most current PCR instruments use volumes in the range 5 to 50 microlitres (or even greater).

It has been proposed, for example in WO 96/41 864 and WO 99/69 168, to heat microscopic-scale reaction volumes via the absorption of electromagnetic radiation. Heating reaction volumes in this way can substantially reduce the time taken to heat a sample from one temperature to another. However, problems arise for larger reaction volumes in arranging that the samples are heated with sufficient uniformity to provide that the integrity of the reaction is preserved.

In this respect, the depth of the reaction volume in the direction of travel of the radiation is limited by the distance over which there is substantially uniform absorption of energy from the incident electromagnetic radiation. Accordingly, to increase the volume which can be uniformly heated, the only option would be to increase the dimensions of the reaction volume perpendicular to the direction of travel of the radiation, and to correspondingly increase the beam width of the incident radiation.

This would result in a significant reduction in the number of reaction volumes that could be practically accommodated side-by-side in an array. Moreover, it will be appreciated by those skilled in the art that to achieve any significant increase in the beam width of the incident radiation would require complex and expensive optics, and is only achievable within certain limits.

According to one aspect of the present invention there is provided a method for controlling the temperature of one or more reaction volumes contained in respective reaction vessel(s) through absorption of electromagnetic energy by a reactant medium of the reaction volume(s), the method comprising the steps of:—

providing one or more reaction volumes in respective reaction vessel(s) such that the reaction volume(s) each have a given depth;

providing at least one source of electromagnetic radiation having selected spectral characteristics; and

directing the electromagnetic radiation into each of the one or more reaction vessel(s) to pass along an absorption path through the reaction volume(s);

selecting the spectral characteristics of the electromagnetic radiation for providing substantially uniform energy absorption by the reactant medium along the full length of said absorption path; and

providing the absorption path for each of the one or more reaction volumes to be greater than or equal to a substantial proportion of a width of that reaction volume in a direction perpendicular to the depth thereof.

By selecting the spectral characteristics of the electromagnetic radiation in this way, relatively deep reaction volumes can be uniformly heated by incident electromagnetic radiation. Thus, radiative heating techniques can be used to obtain uniform heating in larger reaction volumes, with a consequent increase in the rate of heating over the known conduction and convection techniques previously used to heat such reaction volumes.

The present inventors have discovered that by selecting the spectral characteristics of the radiation in relation to the absorption medium, such that the principal wavelength(s) do not coincide with strongly absorbing regions of the absorption spectrum of the reactant medium, the absorption rate can be made to be generally uniform along the absorption path.

In particular, the present invention avoids undesirable surface boiling (with consequent poor thermal transfer through the reaction volumes), which occurs where the principal wavelength(s) of the incident radiation coincide with a strongly absorbing region of the absorption spectrum of the reactant medium.

Moreover, with the present invention, the temperature rise achieved through transfer of energy from the beam of electromagnetic radiation into the reaction volumes is substantially independent of the depth of each reaction volume.

In the specification, the term “reagents” refers to the materials or substances which react together during the chemical/biochemical reaction, the term “reaction medium” refers to the material or substance in which the reagents are mixed or dissolved, and the term “reaction volume” or “reaction mixture” refers to the overall solution or mixture of the reagents and the reaction medium, together with any other materials or substances that may be present.

The spectral characteristics are preferably selected to provide that the electromagnetic radiation at the end of the absorption path through the reaction medium has more than substantially 30% of the energy available at the beginning of the absorption path, or more than substantially 20% of the energy available at the beginning of the absorption path, or more than substantially 15% of the energy available at the beginning of the absorption path.

More preferably, the method further comprises the step of reflecting the electromagnetic radiation which passes through the reaction volume back through the reaction volume, such that the absorption path extends over the full depth of the reaction volume twice.

In this way, the electromagnetic radiation performs a “double pass” through the reaction volume. Thus, more energy from the incident radiation is absorbed by the reaction volume, making the method more efficient. At the same time, the selected spectral characteristics of the radiation provide that the absorption of energy over the full absorption path, i.e., the full double pass, is substantially uniform.

Moreover, reflecting the radiation to perform a double pass through the reaction volume tends to cancel out any minor non-uniformities that may be present. Thus, even greater uniformity of energy absorption (and thus heating) within the reaction volume can be obtained. This can allow for the use of electromagnetic radiation for which the absorption coefficient(s) of the reactant medium is generally higher than for the single pass case, thereby improving the overall efficiency of the process, whilst maintaining uniformity of energy absorption.

The method may further comprise reflecting the electromagnetic radiation which passes through the reaction volume back through the reaction volume, such that the absorption path extends over the depth of the reaction volume more than twice.

In this way, the electromagnetic radiation performs multiple passes through the reaction volume.

The method may further comprise the step of reflecting the electromagnetic radiation which passes through the reaction volume back through the reaction volume.

The absorption path may thus be a zig-zag path through the reaction volume, rather than a single straight line from the surface of the reaction volume to the bottom of the volume (followed by a straight line in the opposite direction in the double-pass case).

In the case where the absorption path extends only once over the depth of the reaction volume, the spectral characteristics of the electromagnetic radiation are preferably selected such that the total absorption of energy over the absorption path is between 5% and 50% of the total energy of the electromagnetic radiation. More preferably, the spectral characteristics of the electromagnetic radiation are selected such that the total absorption of energy over the absorption path is approximately 30% of the total energy of the electromagnetic radiation.

In the case where the absorption path extends twice over the depth of the reaction volume, the spectral characteristics of the electromagnetic radiation are preferably selected such that the total absorption of energy over the absorption path is approximately 80% of the total energy of the electromagnetic radiation.

The optimum percentage absorption of energy from the electromagnetic radiation is the maximum percentage for which sufficient uniformity of energy absorption is achieved. In this way, the efficiency of the process can be maximized, without compromising the uniformity of the heating of the reaction volumes.

In the case where the absorption path extends only once over the depth of the reaction volume, the spectral characteristics of the electromagnetic radiation are preferably selected such that the absorption coefficient of the reactant medium for each of one or more principal wavelengths is between 0.05 cm⁻¹ and 2 cm⁻¹.

More preferably, the spectral characteristics of the electromagnetic radiation are preferably selected such that the absorption coefficient of the reactant medium for each of one or more principal wavelengths is approximately 0.4 cm⁻¹.

In the case where the absorption path extends twice over the depth of the reaction volume, the spectral characteristics of the electromagnetic radiation are preferably selected such that the absorption coefficient of the reactant medium for each of one or more principal wavelengths is between 0.3 cm⁻¹ and 3 cm⁻¹.

More preferably, the spectral characteristics of the electromagnetic radiation are preferably selected such that the absorption coefficient of the reactant medium for each of one or more principal wavelengths is approximately 0.8 cm⁻¹.

These values represent an appropriate range of absorption coefficients in the case where the reactant medium is water, for example. Many chemical or biochemical reactions take place in water (either in a complex solution or as mixture).

The above values also represent an appropriate range of absorption coefficients in the case where the reaction volume has a depth of approximately 10 mm. For deeper reaction volumes, and thus longer absorption path lengths, generally lower absorption coefficients may be required to ensure uniformity of heating over the full absorption path length, whilst for shallower reaction volumes, and thus shorter absorption path lengths, generally higher absorption coefficients can be tolerated.

Preferably, the spectral characteristics of the electromagnetic radiation are selected such that the electromagnetic radiation has a principal wavelength or wavelengths in the range 700 nm to 1200 nm or 700 nm to 1400 nm.

The spectral characteristics of the electromagnetic radiation may be selected such that the electromagnetic radiation has a principal wavelength or wavelengths of approximately 940 nm and/or 980 nm. These values represent appropriate principal wavelengths of commercially available high power laser diodes that are appropriate in the case where the reactant medium is water, for example.

Preferably, the method further comprises the step of conditioning the profile of the electromagnetic radiation between the source and the reaction vessel.

The electromagnetic radiation is preferably conditioned to substantially match a cross-sectional shape of each of the one or more reaction volumes in a plane perpendicular to the absorption path. In this way, the uniformity of heating of over the full width of the reaction volume(s) is improved.

The electromagnetic radiation is preferably conditioned such that the intensity across the profile of the electromagnetic radiation is substantially constant (i.e., there is substantially no intensity variation across its cross-section. Such a profile is termed a “top-hat” profile, and can be achieved using a highly multi-model laser output, or by integrating a low order beam.

More preferably, the electromagnetic radiation is conditioned such that the profile of the electromagnetic radiation includes a lower (or zero) amplitude region in the centre thereof so that substantially no energy is absorbed from the electromagnetic radiation along the central longitudinal axis of the radiation. In this way, convection effects in the central region of the reaction volume balance convection effects at the periphery caused by contact with the vessel walls, so that uniformity of heating over the full width of the reaction volume is improved.

The electromagnetic radiation may be conditioned at its source to achieve the lower (or zero) amplitude region referred to above, or by providing in the path of the radiation between the source of the radiation and the reaction volume(s) non-transmissive element for blocking a portion of the radiation.

Preferably, the selected spectral characteristics comprise two or more peaks (principal wavelengths). In this respect, selecting electromagnetic radiation with more than one principal wavelength will further improve the uniformity of heating of the reaction volume(s).

Preferably, the method further comprises the step of controlling the power delivery rate of the source of electromagnetic radiation.

Controlling the power delivery rate may comprise controlling the power, a pulse duration and/or a pulse length of the electromagnetic radiation emitted by the source.

The power delivery rate is preferably controlled in accordance with a predetermined pattern.

In this way it is possible to cycle the reaction volume(s) between two or more (generally two or three) predetermined temperatures, as required for processes such as PCR.

In preferred embodiments of the present invention, the method further comprises the steps of controlling power delivery rate to:—
a) heat the reaction volume from a first predetermined temperature T1 to a second predetermined temperature T2;
b) maintain the reaction volume at the second predetermined temperature T2 for a predetermined time t1;
c) allow the reaction volume to cool to the first predetermined temperature T1; and
d) repeat steps a) to c) a predetermined number of times.

In this case, the method preferably further comprises the steps of controlling the power delivery rate to:—
b1) allow the reaction volume to cool to a third predetermined temperature T3; and
b2) maintain the reaction volume at the third predetermined temperature T3 for a predetermined time t2;

wherein steps b1) and b2) are performed between steps b) and c).

In preferred embodiments of the invention, the method further comprises the step of directing the electromagnetic radiation sequentially or simultaneously into a plurality of reaction vessels to pass along an absorption path through each of the reaction volumes contained therein.

Preferably, the internal surfaces of the reaction vessel(s) each define a cylindrical volume for containing the respective reaction volume(s), and the method further comprises the step of aligning the reaction vessels relative to the electromagnetic radiation such that a central longitudinal axis of the cylindrical volume defined by the vessel is parallel with the absorption path of the radiation through the reaction medium.

Preferably, the method further comprises the step of sealing the reaction volume within the reaction vessel to prevent or reduce spillage and/or evaporation of the reaction volume.

The absorption path for each of the one or more reaction volumes is preferably greater than or equal to 50% of the width of that reaction volume in a direction perpendicular to the depth thereof.

More preferably, the absorption path for each of the one or more reaction volumes is greater than or equal to the width of that reaction volume in a direction perpendicular to the depth thereof.

In preferred embodiments of the present invention, the method further comprises the step of mounting the reaction vessel(s) to be in thermal contact with a support member having a large thermal mass relative to the reaction volume(s).

Preferably, the method further comprises the step of maintaining the temperature of the support member at a predetermined temperature T1.

The electromagnetic radiation may be directed into the reaction vessel(s) to be incident on the surface of the reaction volume(s) at an angle of incidence substantially equal to the Brewster angle for the reaction volume(s). The use of polarized electromagnetic radiation will improve the optical coupling.

The electromagnetic radiation may be directed into the reaction vessel(s) to be incident on the surface of the reaction volume(s) at an angle of incidence substantially equal to the angle required for total internal reflection of the radiation within the reaction volume.

In preferred embodiments of the present invention, the method further comprises the step of directing further electromagnetic radiation to and from the reaction volume for monitoring the progress of a reaction in the reaction volume.

According to another aspect of the present invention there is provided an apparatus for performing the method as claimed in any preceding claim.

Preferably, the one or more reaction vessels may each comprise a lid for sealing the respective reaction volume(s) therein.

Preferably, the reaction vessel comprises a reflective base for reflecting substantially all of the electromagnetic radiation which passes through the reaction volume back through the reaction volume. Alternatively, a reflective element which is substantially totally reflective to the beam of electromagnetic radiation may be provided outside the walls of the container for reflecting the beam of electromagnetic radiation.

The internal surface of the lid may be partially reflective to the beam of electromagnetic radiation for partially reflecting the beam of electromagnetic radiation back into the reaction volume.

The internal surfaces of the reaction vessel may be at least partially reflective to the beam of electromagnetic radiation.

Preferably, the reaction vessel(s) each have internal surfaces which define a substantially cylindrical volume for containing the respective reaction volume(s).

The apparatus preferably further comprises a support member having a large thermal mass relative to the reaction volume(s) for contacting the reaction vessel(s).

Preferably, the reaction vessel(s) each have external surfaces which are tapered to fit in correspondingly shaped receiving hole(s) in the support member. This improves the thermal contact between the block and the reaction volume(s) contained in the reaction vessel(s).

The external surfaces of the reaction vessel(s) may be provided with a screw thread for engaging with a correspondingly threaded portion in the respective receiving hole(s).

In preferred embodiments of the present invention, the apparatus comprises means for applying the electromagnetic radiation sequentially or simultaneously to a plurality of reaction volumes. The means for applying the electromagnetic radiation sequentially or simultaneously can comprise optical scanning and/or other optical components including mirrors, lenses, conduits and the like, as required to distribute the electromagnetic radiation among the reaction volumes.

The apparatus of the present invention may comprise diffractive optics, adaptive optics, hollow waveguides and/or fibre optics for directing the electromagnetic radiation sequentially or simultaneously into a plurality of reaction vessels.

The source of electromagnetic radiation may comprise one or more lasers and/or one or more light emitting diodes.

In preferred embodiments of the present invention, a common optical channel may be provided for both directing the electromagnetic radiation into the reaction volume to promote a chemical reaction therein, and to direct further electromagnetic radiation to and from the reaction volume to monitor the progress of said chemical reaction.

Embodiments of the present invention are described below with reference to the accompanying drawings in which:—

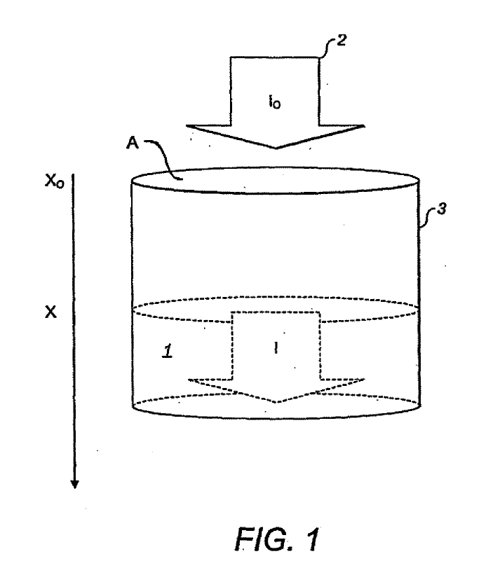

FIG. 1 illustrates the theory behind the present invention;

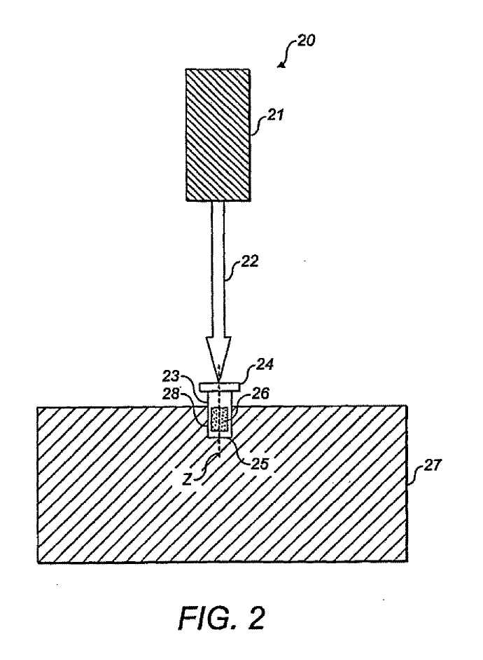

FIG. 2 illustrates an embodiment of the present invention in simplified form;

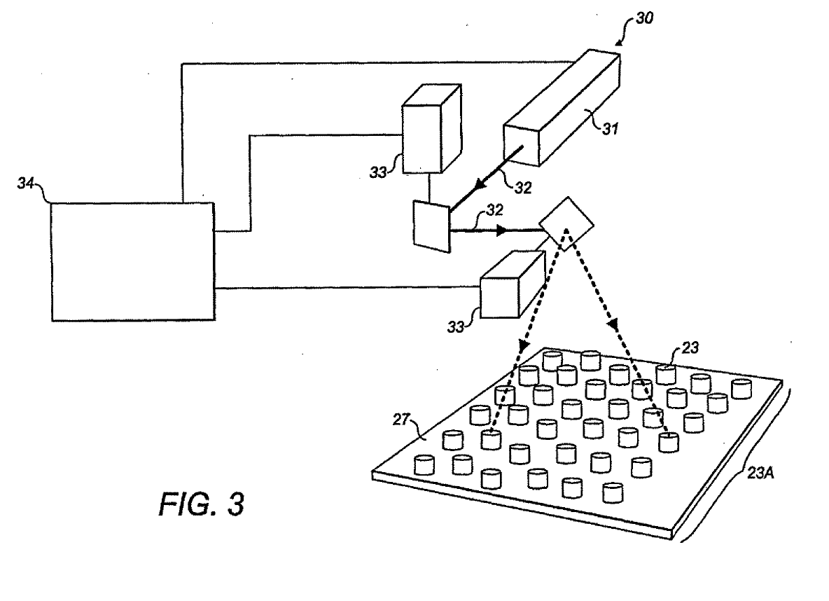

FIG. 3 is a schematic illustration of apparatus for serially scanning an array of reaction volumes in accordance with one embodiment of the invention;

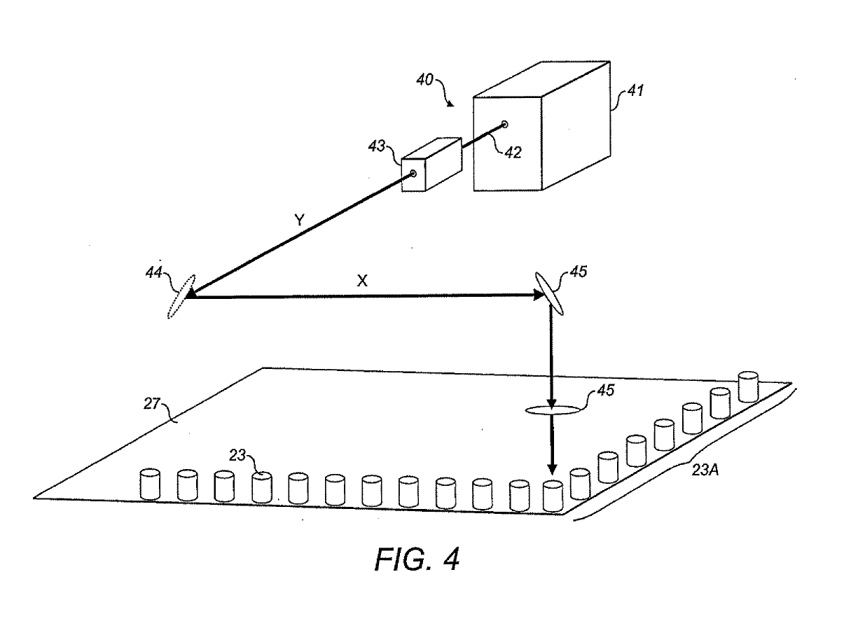

FIGS. 4 to 6 are schematic illustrations of apparatus for serially scanning an array of reaction volumes in accordance with alternative embodiments of the invention;

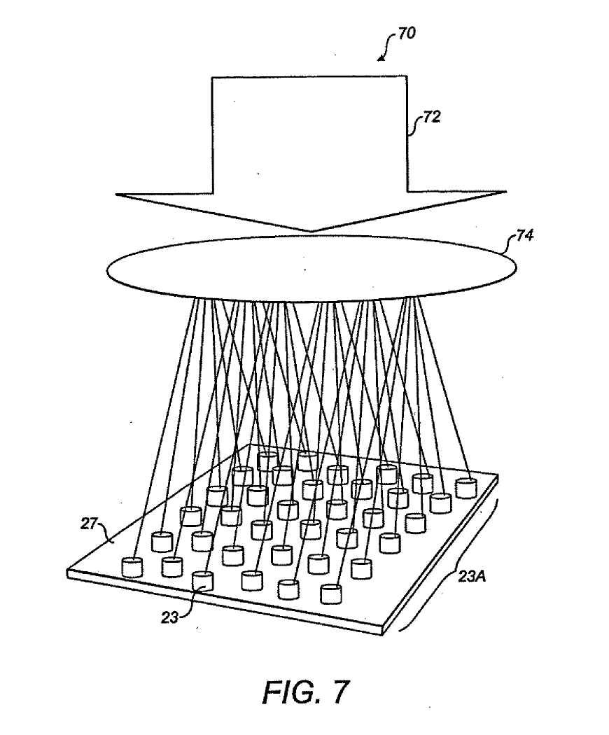

FIG. 7 is a schematic illustration of apparatus for simultaneously scanning an array of reaction volumes in accordance with a further embodiment of the present invention; and

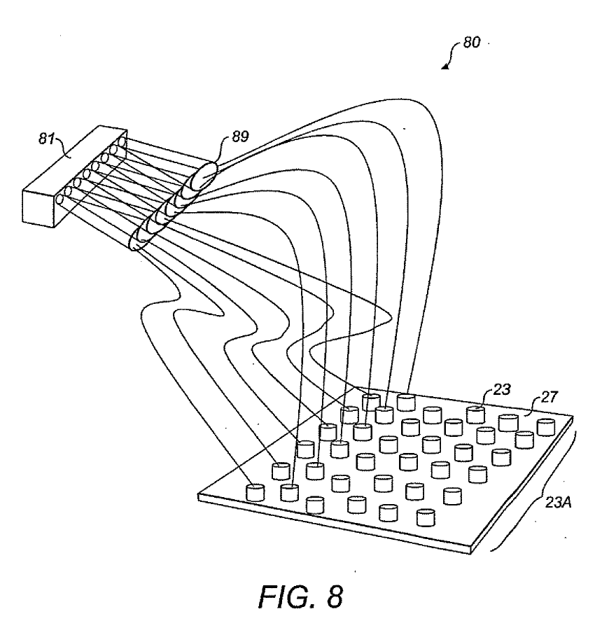

FIG. 8 is a schematic illustration of apparatus for simultaneously scanning an array of reaction volumes in accordance with yet another embodiment of the present invention.

In the drawings, elements common to the various embodiments illustrated are given common reference numerals.

The theory behind the present invention will now be explained with reference to FIG. 1. It is convenient for the purpose of analysing the heating of reaction volumes by incident electromagnetic radiation to consider a cylindrical volume 1 with cross-sectional area A, as shown in FIG. 1.

It is well known that electromagnetic radiation 2 passing along an absorption path through a medium is governed by equation (1).

I=I₀exp[−bX] (1)

where I is the intensity at a distance X along the absorption path measured from an origin at the surface of the medium where X=0 and I=I₀, and b is the absorption coefficient (often with units cm⁻¹).

The fractional intensity is then given by equation (2).

I/I₀=exp[−bX] (2)

It will thus be appreciated that the intensity (or fractional intensity) of the radiation decreases exponentially as it passes through the medium, and that the rate at which the intensity decreases (i.e., the rate at which energy is absorbed by the medium) over a given distance depends on the value of the absorption coefficient. In particular, the present inventors have discovered that by selecting a sufficiently low absorption coefficient, the rate of absorption of energy over a given distance from X=0 can be made sufficiently uniform to provide substantially uniform heating of the medium over that distance.

To illustrate this point, example values of the fractional intensity for a single-pass of electromagnetic radiation through a reaction volume with a depth of 10 mm are tabulated for various values of b and X in Table 1. Example values of the fractional intensity for a double-pass of electromagnetic radiation through a reaction volume with a depth of 10 mm are tabulated for various values of b and X in Table 2.

TABLE 1

| | Absorption coefficient = | | Absorption coefficient = | |
|---|---|---|---|---|
| | 0.29 cm⁻¹ (water @ 940 nm) | | 0.43 cm⁻¹ (water @ 980 nm) | |
| | Fractional | Fractional | Fractional | Fractional |
| | intensity | intensity | intensity | intensity |
| X in | remaining | absorped in | remaining | absorped in |
| mm | at X | each mm | at X | each mm |
| 1.0 | 0.971 | 0.029 | 0.958 | 0.042 |
| 2.0 | 0.944 | 0.028 | 0.918 | 0.040 |
| 3.0 | 0.917 | 0.027 | 0.879 | 0.039 |
| 4.0 | 0.890 | 0.026 | 0.842 | 0.037 |
| 5.0 | 0.865 | 0.025 | 0.807 | 0.035 |
| 6.0 | 0.840 | 0.025 | 0.773 | 0.034 |
| 7.0 | 0.816 | 0.024 | 0.740 | 0.033 |
| 8.0 | 0.793 | 0.023 | 0.709 | 0.031 |
| 9.0 | 0.770 | 0.023 | 0.679 | 0.030 |
| 10.0 | 0.748 | 0.022 | 0.651 | 0.029 |
| Total | | 0.25 | | 0.35 |

Absorption coefficient =
1.2 cm⁻¹ (water @ 1200 nm)

| | Fractional | Fractional |
|---|---|---|
| | intensity | intensity |
| X in | remaining | absorped in |
| mm | at X | each mm |
| 1.0 | 0.887 | 0.113 |
| 2.0 | 0.787 | 0.100 |
| 3.0 | 0.698 | 0.089 |
| 4.0 | 0.619 | 0.079 |
| 5.0 | 0.549 | 0.070 |
| 6.0 | 0.487 | 0.062 |
| 7.0 | 0.432 | 0.055 |
| 8.0 | 0.383 | 0.049 |
| 9.0 | 0.340 | 0.043 |
| 10.0 | 0.301 | 0.038 |
| Total | | 0.70 |

TABLE 2

| | Absorption | Absorption | Absorption |
|---|---|---|---|
| | coefficient = | coefficient = | coefficient = |
| | 0.29 cm⁻¹ (water | 0.43 cm⁻¹ (water | 1.2 cm⁻¹ (water |
| | @ 940 nm) | @ 980 nm) | @ 1200 nm) |
| | Fractional | Fractional | Fractional |
| | intensity | intensity | intensity |
| X in | absorped in | absorped in | absorped in |
| mm | each mm | each mm | each mm |
| 1.0 | 0.045 | 0.061 | 0.125 |
| 2.0 | 0.045 | 0.060 | 0.113 |
| 3.0 | 0.044 | 0.059 | 0.104 |
| 4.0 | 0.044 | 0.058 | 0.095 |
| 5.0 | 0.044 | 0.058 | 0.089 |
| 6.0 | 0.044 | 0.057 | 0.083 |
| 7.0 | 0.044 | 0.057 | 0.079 |
| 8.0 | 0.044 | 0.056 | 0.076 |
| 9.0 | 0.043 | 0.056 | 0.074 |
| 10.0 | 0.043 | 0.056 | 0.072 |
| Total | 0.44 | 0.58 | 0.91 |

If the input radiation is uniform in intensity and has a circular cross-section that exactly matches the cross-sectional area A of the cylinder upon which it is incident, equation (1) can be multiplied by the area A to give the incident beam power, P. Then, for a pulse of radiation of duration t seconds, the fractional intensity describes the fraction of incident energy (fractional energy) that continues to propagate through the medium at any value of X.

It will be appreciated that the percentage energy absorbed in the medium before reaching X is equal to (1−fractional energy)×100. Thus, referring to the values in Table 1, for an absorption coefficient of 0.29 cm⁻¹ or 0.43 cm⁻¹, it can be seen that energy is substantially uniformly absorbed over an absorption path of (at least) 10 mm through the reaction volume 1. In contrast, for an absorption coefficient of 1.2 cm⁻¹, the percentage energy absorbed over the first millimetre along the absorption path is approximately three times the percentage energy absorbed over the tenth millimetre.

From Table 2, it can be seen that the double-pass configuration significantly assists with uniform heating of the reaction volume. Although, with the present invention, the energy distribution along the full length of the absorption path of the radiation through the reaction volume is substantially uniform, it will be appreciated that there is a slight decrease in the fractional energy absorbed as the radiation progresses through the reaction volume. By reflecting the radiation back through the reaction volume in the opposite direction, this slight decrease is substantially compensated for.

By selecting the spectral characteristics of the incident electromagnetic radiation in relation to the absorption spectrum of the reactant medium of the reaction volume, it is possible to select radiation with a principal wavelength for which the absorption coefficient b of the reactant medium is relatively low. Energy from the incoming beam will then be distributed uniformly through the depth of the reaction volume 1.

In the example values tabulated in Table 1, the absorption coefficients 0.29 cm⁻¹, 0.43 cm⁻¹ and 1.2 cm⁻¹ respectively correspond to wavelengths of 940 nm, 980 nm and 1200 nm in liquid water. Accordingly, electromagnetic radiation having principal wavelength(s) at 940 nm and/or 980 nm can be used to uniformly heat a reaction volume for which the absorption path length is up to at least 10 mm.

In practice, transverse effects due to thermal diffusion gradients established between the heated medium and the cooler walls of its containing vessel 3 will affect the degree of uniformity. However, despite these effects, it is clear that lower absorption values result in more uniform heating for columnar sample volumes as compared with high absorption values.

Importantly, since a lower absorption coefficient results in more uniform heating of the reaction volume the optimum absorption regime requires that the principal wavelength(s) of the incident electromagnetic radiation should not be coincident with strongly absorbing regions in the absorption spectrum of the reactant medium.

If a volume error is made, for example an extra 0.5 mm depth of the PCR mixture is introduced into the reaction vessel 3, it might be thought that the rate of temperature rise would be reduced owing to the extra volume of PCR mixture to be heated. However, since there is an excess of energy in the radiation, the addition of the extra volume simply results in a corresponding increase in the fractional absorption.

For example, if the reaction vessel is filled with PCR mixture to a depth of 5.5 mm instead of 5.0 mm, this corresponds to a 1 mm increase in the absorption path length for a double pass of the radiation through the reaction volume. With an absorption coefficient of 0.43 cm⁻¹, this additional 1 mm results in the fractional absorption increasing from 0.35 to 0.38 (using equation (2)). Hence, a 10% increase in depth is balanced by a 10% increase in the energy absorbed. By the same argument, reductions in volume mean less energy is absorbed. It can therefore be seen that the temperature rise is substantially independent of errors in volume.

The only mechanisms that strongly affect the rate of temperature rise are the value of the input energy (i.e., the laser's power stability and the constancy of its pulse “on time”), conduction out of the reaction vessel and the value of the water absorption (which is inherently constant).

An embodiment of the present invention will now be described with reference to FIG. 2, which shows apparatus 20 embodying the present invention.

A high powered diode laser 21 (more specifically, a 100 Watts CW @ 980 nm, for example, a pulsable diode laser bar) emits a beam of electromagnetic radiation 22. The laser's output is made circular and integrated to provide a uniform “top-hat” intensity profile, and then collimated to a diameter of 2 mm. The collimated beam is then further conditioned to provide a lower (or zero) amplitude region in the centre of the beam profile. The conditioning of the beam profile may be achieved using any one of a number of methods which will be readily apparent to those skilled in the art. Aspheric optics, anamorphic prism pairs and beam integrators are commercially available for this purpose. With regard to providing the lower (or zero) amplitude region in the centre of the beam profile, it will be appreciated that instead of conditioning the radiation at its source, this profile may be achieved by providing an at least partially non-transmissive element (for example, an at least partially absorbing element or an at least partially reflective element) in the path of the radiation for at least partially blocking a portion of the radiation along the central longitudinal axis thereof. Conveniently, the at least partially non-transmissive element could be provided on the lid 24 of the reaction vessel 23 referred to below.

By integrating and collimating the beam 22, the beam profile can be made to match the reaction volume's cross-sectional area so as to achieve more uniform heating across the full width of the reaction volume. By providing a lower (or zero) amplitude region in the middle of the beam profile, there will be less (or no) electromagnetic energy absorbed along the central longitudinal axis of the reaction volume 26. As a result, convection currents are generated in the centre of the reaction volume which balance convection currents at the periphery caused by conduction through the vessel walls, to provide substantially uniform heating across the full width of the reaction volume.

The apparatus of the present embodiment comprises an array of separate PCR reaction vessels 23, only one of which is shown in FIG. 2 for clarity.

Each PCR reaction vessel 23 is a small plastic vial whose internal surfaces define a cylindrical volume which has a central longitudinal axis Z and a circular cross-section. Each vessel 23 has a tight fitting lid 24. The material from which the vessel 23 and the lid 24 are formed is chosen to be substantially transparent to 980 nm electromagnetic radiation. In practice, the material from which the vessel is formed would be a common plastic material, most of which are transparent to 980 nm radiation. Thus, the covering lid 24 prevents excessive evaporation of the reaction volume 26, but allows transmission of the incoming laser beam 22.

In contrast, a base 25 of the vessel 23 is coated to be substantially totally reflective to 980 nm electromagnetic radiation.

The vessels 23 are filled to 5 mm deep with PCR mixture containing the necessary reagents for a biochemical PCR procedure mixed or dissolved in water. This PCR mixture constitutes the reaction volume (sample) 26.

In use, the beam of electromagnetic radiation 22 emitted by the laser 21 such is directed (either sequentially or simultaneously) through the transparent lid 24 of each vessel 23 into the reaction volume 26 contained therein. The reaction vessels 23 are aligned such that the axis Z for each vessel is parallel to an absorption path of the radiation through the reaction volume 26. The beam of electromagnetic radiation 22 will then travel downwardly through the reaction volume 26, will be reflected by the reflective coating of the base 25, and will then travel upwardly through the reaction volume 26. The absorption path of the radiation thus has a total length equal to twice the depth of the reaction volume, i.e., 10 mm.

Although in the present embodiment the reflective element is a reflective coating applied to the base 25 of the reaction vessel 23, the same effect can be achieved by means of a reflective element that is substantially totally reflective to 980 nm radiation situated vertically below the vessel 23.

Radiation not absorbed within the reaction volume may be safely directed into an absorbing medium by one of a number of methods which will be readily apparent to those skilled in the art.

The reaction vessel 23 is mounted into a support element in the form of a shaped block (body/thermal mass) 27. For clarity, the block 27 is shown in FIG. 2 to have a single receiving hole 28 for accommodating a single reaction vessel 23. In reality, the block 27 is shaped to accommodate the array of reaction vessels, with each reaction vessel 23 accommodated in an individual receiving hole 28. The mounting of the reaction vessels 23 in the block 27 is such that good thermal contact is made between the vessels 23 and the block 27. This can be achieved by making the external surface of the wall of the reaction vessels 23 slightly tapered to match a similar taper in each receiving hole 28 in the block 27 (not shown in FIG. 2). The mating surfaces of each reaction vessel 23 and its associated receiving hole 28 may be screw threaded to provide additional security to the mounting, and increased thermal transfer. The internal surface of the wall of the reaction vessels 23 remain cylindrical, despite the tapering and/or threaded portions on the external surface.

The block 27 is much larger than the reaction vessels 23 and has a thermal mass that is significantly greater than that of the reaction vessels 23 and the reaction volumes 26 therein. The block 27 incorporates a Peltier effect element that allows it to be cooled and heated in a controlled manner so as to maintain a steady temperature T1, for example, approximately 50° C. Such blocks, with good thermal conductivity are commonly made from metals or ceramics and are widely available commercially.

Alternatively, the block 27 may be cooled and heated by a flow of hot or cold air. In this case, the air would be delivered in a controlled manner to maintain a steady temperature T1, for example, approximately 50° C.

The vessels 23 quickly reach thermal equilibrium with the body at temperature T1.

The laser 21 is switched on for a period of time so that the temperature of each reaction volume 26 is increased to a temperature T2, for example, approximately 95° C. The power delivery rate of the laser 21 can be adjusted to control the speed at which the reaction volume is heated to T2. Once the reaction volume has been heated to T2, the power delivery rate is lowered so that the reaction volume is maintained at the temperature T2 against the tendency of the reaction volume to cool to T1 through conduction of heat into the block 27 for a desired time period t1. In this connection, the power delivery rate of the laser may be adjusted by controlling a pulse rate and/or intensity of the beam emitted by the laser.

The incident radiation 22 is then removed to allow the reaction volumes 26 to cool to a temperature T3 that is higher than T1 but lower than T2, for example, approximately 70° C. When the temperature of the reaction volume has cooled to T3, the laser is then switched on again with a lower power delivery rate to maintain the reaction volume at T3 for a desired time period t3. Finally, the incident radiation is again removed, to allow the temperature of the reaction volume to drop quickly back to T1.

By repeating these steps, the reaction volumes 26 can be repeatedly thermally cycled with only very short time periods spent in heating the reaction volumes, so that the overall process time can be reduced.

In order to provide that the reaction volumes 26 are heated, cooled and maintained at the required temperatures, a control sample (not shown) is provided adjacent one of the plurality of reaction volumes. The control sample is equipped with a temperature monitoring device such as a thermocouple or RTD (Resistance Temperature Detector) to directly monitor the temperature of the control sample. The output from the temperature monitoring device is connected to a controller (not shown) for controlling the power delivery rate of the laser (for example by controlling the laser power or laser pulse rate) and/or the means for heating and cooling the block 27 to enable the required temperatures to be obtained for the required time periods.

The following analysis assumes that temperature distribution within the reaction volume 26 is uniform. Whilst, in practice, this is not strictly the case, it can be shown that a significant volume fraction (approximately 70%) of the reaction volume 26 will experience a substantially uniform temperature increase and decrease as the reaction volume is thermally cycled. This allows for effective thermal cycling in the case of PCR procedures, as well as other procedures.

In the present embodiment, the volume of the PCR mixture is π(1)²×5 mm³=5π mm³. For the purposes of the following analysis, the reaction volume 26 can, to a very good approximation, be regarded as consisting of water, so that its mass is 5π×10⁻⁶ kg. Using a simple energy balance gives equation (3).

mass (m)×specific heat capacity for water (b)×change in temperature (ΔT)=input power (P)×pulse duration (t)×fractional absorption (3)

For incident radiation 22 with a principal wavelength of 980 nm, equation (4) is obtained from equation (3) (see Table 1).

m×4181 J/kg/C×ΔT=P×t×0.35 (4)

For PCR, the temperature of the reaction volume 26 needs to be raised from around 50° C. to around 95° C. for the denaturing of DNA. This means that ΔT=45° C. Rearranging equation (4), and setting m=5π×10⁻⁶ kg and P=100 Watts, it can be seen that t=84 milliseconds=0.084 seconds.

Neglecting the time taken to manipulate the laser beam 22 around the plurality of reaction volumes 26, the time taken to denature N reaction volumes (each having a volume of approximately 16 microlitres) is N×0.084 seconds, i.e., just over 8 seconds for 96 reaction volumes. This represents a considerable improvement over existing methods of heating. In conventional terminology, the present invention achieves heating rates of between 100 and 500° C./second. The rates achieved by existing methods are between 1 and 5° C./second for Peltier heaters and 10° C. per second for hot air systems.

In the following analysis it is assumed that the whole reaction volume 26 cools uniformly through contact with the block 27. Although this is not precisely the case in practice, it has been observed that a significant volume fraction of the reaction volume will maintain a near uniform temperature distribution as the reaction volume cools.

The rate of cooling can be evaluated in terms of energy lost from the reaction volume 26. A 0.5° C. fall in temperature of the 5π mm³ reaction volume corresponds to an energy loss of 0.033 Joules=33 mJ.

Arbitrarily small “packets” of energy can be input using the laser 21. For example, a 33 mJ packet corresponds to a pulse of 330 microseconds duration from a 100 W laser output. Allowance must be made for the fact that only 35% of this will be absorbed. Hence, a pulse of around 1 millisecond from the 100 W laser output is required to compensate for a 0.5° C. fall in temperature. It can thus be seen that by adjusting the power transfer rate of the laser, i.e., by adjusting pulse duration, pulse rate and/or laser power, the temperature of the reaction volume 26 can be accurately controlled to maintain a desired temperature for an arbitrary period of time.

In practice a smaller “packet” could be used to control the temperature with a resolution much better than 0.5° C.

In many applications, there will be a plurality of reaction volumes, each of which needs to be addressed individually. The number of reaction volumes that can be maintained at an intermediate temperature for an arbitrary time depends on the rate of temperature fall (i.e., energy loss) for each reaction volume, the laser power available and the time taken to address and irradiate each sample with a “packet” of energy from the laser beam.

If, for example, it is assumed that the reaction volume 26 cools at a rate of 2° C./second, approximately 0.5 seconds is available between each time a sample is addressed in order to maintain a 1° C. reaction volume temperature accuracy.

A 100 W laser 21 delivered to a 12×8 array of reaction vessels each with a cross-sectional area of around 40×60 mm² by means of a beam 22 which can move at 640 mm/second over a total path of about 240 mm is capable of addressing each sample within 0.5 seconds, thus making it possible to maintain a desired temperature to an accuracy of +/−1° C. against a cooling rate of 2° C./second.

The technique of delivering small energy “packets” could be used, if necessary or desirable, to maintain reaction volumes at any temperature that is higher than the temperature of the block 27 for an arbitrary time. For example, the denaturing temperature (95° C.) and extension temperature (70° C.) for PCR. Although a cooling rate of 2° C./second has been assumed above, it will be appreciated by those skilled in the art that the true cooling rate will depend on the temperature of reaction volume, the temperature of the block 27, and the thermal conductivity therebetween.

The delivery of radiation “packets” to all reaction volumes simultaneously (as might be achieved using a diffractive lens or mirror, or a bundle or array of fibre optic cables) will provide scope for greater accuracy in maintaining a given temperature, since the time required to serially address each sample is removed.

Various arrangements for dealing with multiple reaction volumes are illustrated in FIGS. 3 to 8.

FIG. 3 is a schematic illustration of apparatus 30 for serially scanning an array of reaction volumes 23A. A static laser 31 emits a laser beam 32 that may be homogenised and partially focused. The laser beam 32 is scanned or directed, serially over an array of small reaction volumes 23A. The beam 32 from the static laser 31 can, therefore, address any individual reaction volume 23 from the array thereof by means of a CNC (Computer Numerical Control) galvanometric scanner 33. A motion controller 34 (for example, a personal computer) directs the beam 32. The motion controller 34 controls both the laser power and the two axes of the galvanometric scanner 33. Such devices are well know to those familiar with laser material processing and are commercially available. The scanner need not be galvanometric (for example, it could be acousto-optic) and it could be replaced by a system of controllable mirrors in a Cartesian arrangement as shown schematically in FIG. 4.

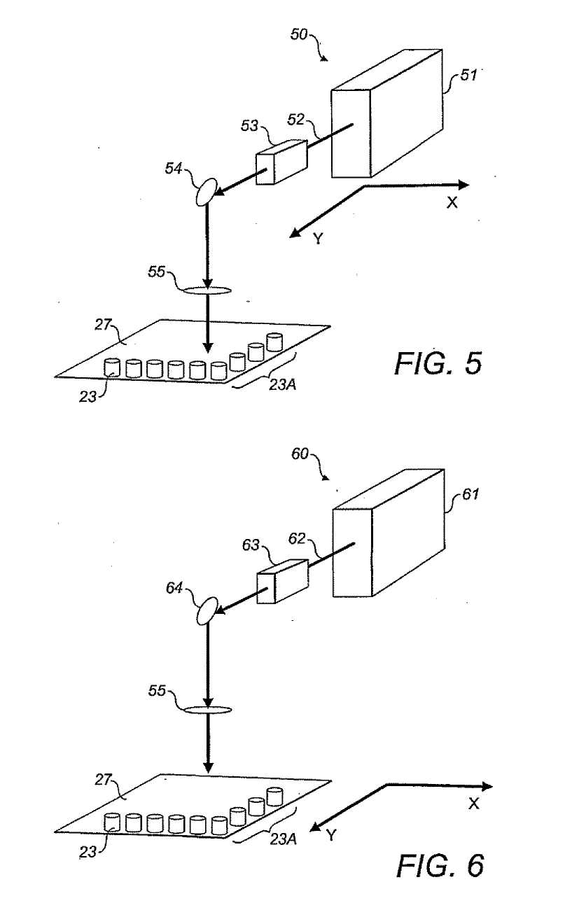

Alternatively, the laser or the array could be moved relative to one another in a controllable fashion as shown, for example, in FIGS. 5 and 6.

In the apparatus 40, 50 and 60 respectively shown in FIGS. 4, 5 and 6 a beam of electromagnetic radiation 42, 52, 62 from a laser 41, 51, 61 is integrated and collimated by a beam conditioning device (collimator and integrator) 43, 53, 63. The beam is then bent through 90 degrees by a full reflector mirror 44, 54, 64 and directed towards a transmissive lens 45, 55, 65 (optional).

A 12×8 array 23A of reaction vessels 23 are provided on a workpiece (block) 27, as described above. In the apparatus of FIG. 4, the flying optic X and Y axes are movable by a system of controllable mirrors under CNC Control. In the apparatus of FIG. 5, the laser is moveable in two dimensions (along the X and Y axes shown in FIG. 5) under CNC control. In the apparatus of FIG. 6, the workpiece 27 is provided on a moving table (not shown) which is movable in two dimensions (along the X and Y axes shown in FIG. 6) under CNC control. In each case the beam of electromagnetic radiation transmitted through the transmissive lens (or from the full reflector mirror in cases where the transmissive lens is not present) is caused to be incident on the reaction mixture in each of the reaction vessels 23 in turn.

All of these techniques produce an equivalent result as regards the scanning of a beam of energy over an array of samples, but one or other of the techniques may be preferred in any given set of practical circumstances.

By switching the laser on and off in a timely manner to produce the required levels of incident average power, temperature cycling of a number of reaction volumes can be performed. If the number is not too large then the reaction volumes can be cycled in a quasi-simultaneous manner provided that a beam generator of adequate output power (greater than 10 Watts) and a fast enough beam director are available.

As explained previously, it is straightforward to increase the accuracy of temperatures to be achieved or maintained within a temperature cycle by incorporating sample volumes equipped with temperature transducers at locations within a sample array. These control samples will enable system parameters to be controlled so as to increase temperature accuracy over that which can be achieved by “dead reckoned” or so-called open loop parameters.

FIGS. 7 and 8 are schematic representations of apparatus for effecting the simultaneous heating of a number of small reaction volumes set out in an array, for example, a 2-D square array.

FIG. 7 schematically shows a suitably expanded laser beam 72 incident on a suitable diffractive lens 74 so as to provide multiple individual beams (or foci) matching the array 73. In this way simultaneous heating of a number of reaction volumes is possible and hence rapid thermal cycling is also possible. In this case the laser (not shown in FIG. 7) providing the input beam might be, for example, a solid state Infra-Red laser.

The design of the necessary diffractive optics would be straightforward for a person skilled in the art to implement.

FIG. 8 schematically shows a linear array of diode lasers 81 (commonly called a diode bar) split up into a larger number of beams by a bundle of fibre optics 89 to accomplish the same task as described with reference to FIG. 7. The beams from the diode array 81 are allowed to propagate to create a quasi-rectangular beam where they are significantly overlapping due to their inherent individual divergences. In fact, a diode laser array or number of arrays might can be used without fibre optic splitting to achieve the same outcome. Again, the implementation of these techniques would be straightforward for a person skilled in the art to implement.

The progress of a chemical reaction is often monitored by optical methods such as measuring the intensity of fluorescence from a dye species that has attached uniquely to a product of the chemical reaction. Other optical methods of monitoring include the measurement of light scattering unique to the product of the chemical reaction. The present invention offers the opportunity to use a common optical channel for both heating to promote the reaction and monitoring the subsequent progress by directing the appropriate electromagnetic radiation into and from the reaction volume.

The present invention has been described above in the context of PCR procedures. However, as will be readily apparent to a person skilled in the art, the present invention will also be applicable to a wide variety of other procedures involving thermally cycling reaction volumes, or indeed any procedures where accurate temperature control of reaction volumes is required.

The present invention has been described in terms of a case where the incident electromagnetic radiation is incident on the surface of the reaction volume at right angles thereto. However, the radiation may be incident on the surface at other angles thereto. In these cases, the side walls of the reaction vessel may be made at least partially reflective such that the radiation may be reflected internally within the vessel to follow a zig-zag path through the reaction vessel.

1-54. (canceled)

55. A method for controlling the temperature of one or more reaction volumes of a reaction mixture contained in respective reaction vessel(s) through absorption of electromagnetic energy by a reactant medium of the reaction mixture, the method comprising:
providing a reaction mixture wherein the reactant medium is water;
arranging one or more reaction volumes of the reaction mixture in respective reaction vessel(s);
arranging at least one source of electromagnetic radiation to direct electromagnetic radiation into the or each reaction vessel along an absorption path through the reaction volume; and
selecting the spectral characteristics of said electromagnetic radiation to have a principle wavelength or wavelengths in the range 700 nm to 1200 nm to provide substantially uniform energy absorption by the reactant medium along the full length of the absorption path.

56. A method as claimed in claim 55, further comprising reflecting the electromagnetic radiation which passes through the reaction volume back through the reaction volume, such that the absorption path extends over the full depth of the reaction volume at least twice.

57. A method as claimed in claim 55, wherein the absorption path extends only once over the depth of the reaction volume, and the spectral characteristics of the electromagnetic radiation are selected such that the total absorption of energy over the absorption path is between 5% and 50% of the total energy of the electromagnetic radiation.

58. A method as claimed in claim 56, wherein the spectral characteristics of the electromagnetic radiation are selected such that the total absorption of energy over the absorption path is approximately 80% of the total energy of the electromagnetic radiation.

59. A method as claimed in claim 55, wherein the spectral characteristics of the electromagnetic radiation are selected such that the electromagnetic radiation has a principal wavelength or wavelengths of approximately 940 nm and/or 980 nm.

60. A method as claimed in claim 55, wherein the electromagnetic radiation is conditioned such that the profile of the electromagnetic radiation includes a lower (or zero) amplitude region in the centre thereof so that substantially no energy is absorbed from the electromagnetic radiation along the central longitudinal axis of the radiation.

61. A method as claimed in claim 60, wherein the electromagnetic radiation is conditioned by providing in the path of the radiation between the source of the radiation and the reaction volume(s) non-transmissive element for blocking a portion of the radiation.

62. A method as claimed in claim 55, wherein the selected spectral characteristics comprise two or more peaks (principal wavelengths).

63. A method as claimed in claim 55, further comprising the steps of controlling power delivery rate to:
a) heat the reaction volume from a first predetermined temperature T1 to a second predetermined temperature T2;
b) maintain the reaction volume at the second predetermined temperature T2 for a predetermined time t1;
c) allow the reaction volume to cool to the first predetermined temperature T1; and
d) repeat steps a) to c) a predetermined number of times.

64. A method as claimed in claim 63, further comprising the steps of controlling the power delivery rate to:
b1) allow the reaction volume to cool to a third predetermined temperature T3; and
b2) maintain the reaction volume at the third predetermined temperature T3 for a predetermined time t2;
wherein steps b1) and b2) are performed between steps b) and c).

65. A method as claimed in claim 55, wherein the absorption path for each of the one or more reaction volumes is preferably greater than or equal to 50% of the width of that reaction volume in a direction perpendicular to the depth thereof.

66. A method as claimed in claim 65, wherein the absorption path for each of the one or more reaction volumes is greater than or equal to the width of that reaction volume in a direction perpendicular to the depth thereof.

67. A method as claimed in claim 55, wherein the electromagnetic radiation is directed into the reaction vessel(s) to be incident on the surface of the reaction volume(s) at an angle of incidence substantially equal to the Brewster angle for the reaction volume(s)

68. A method as claimed in claim 55, wherein electromagnetic radiation is directed into the reaction vessel(s) to be incident on the surface of the reaction volume(s) at an angle of incidence substantially equal to the angle required for total internal reflection of the radiation within the reaction volume.

69. Apparatus for performing the method as claimed in claims 55.

70. Apparatus as claimed in claim 69, wherein the reaction vessel comprises a reflective base for reflecting substantially all of the electromagnetic radiation which passes through the reaction volume back through the reaction volume.

71. Apparatus as claimed in claim 69, wherein a reflective element which is substantially totally reflective to the beam of electromagnetic radiation is provided outside the walls of the container for reflecting the beam of electromagnetic radiation.

72. Apparatus as claimed in claim 69, wherein the internal surface of the lid is partially reflective to the beam of electromagnetic radiation for partially reflecting the beam of electromagnetic radiation back into the reaction volume.

73. Apparatus as claimed claim 69, wherein the internal surfaces of the reaction vessel may be at least partially reflective to the beam of electromagnetic radiation.

74. Apparatus as claimed in claim 69, wherein a common optical channel is provided for both directing the electromagnetic radiation into the reaction volume to promote a chemical reaction therein, and to direct further electromagnetic radiation to and from the reaction volume to monitor the progress of said chemical reaction.
